(19) United States
(12) Patent Application Publication
(10) Pub. No.: US 2019/0249045 A1
(43) Pub. Date: Aug. 15, 2019

(54) HOT-MELT ADHESIVE AGENT

(71) Applicant: HENKEL AG & CO. KGAA, Duesseldorf (DE)

(72) Inventor: Chiho FUJITA, Osaka (JP)

(21) Appl. No.: 16/391,546

(22) Filed: Apr. 23, 2019

Related U.S. Application Data
(63) Continuation of application No. PCT/JP2017/036274, filed on Oct. 5, 2017.

(30) Foreign Application Priority Data
Oct. 26, 2016 (JP) 2016-209655

(51) Int. Cl. C09J 153/02; A61F 13/56; C09J 7/35

(52) U.S. Cl. CPC C09J 153/025; C09J 7/35; A61F 13/5605

(57) ABSTRACT

To provide a hot-melt adhesive agent which has excellent retention strength and anti-fragrance property, and shows little residual adhesive and unpleasant odor. A hot-melt adhesive agent having a thermoplastic block copolymer (A), the (A) being a copolymer of vinyl class aromatic hydrocarbons and conjugated diene compounds, wherein the thermoplastic block copolymer (A) comprises an unhydrogenized triblock type styrene isoprene block copolymer (A1) and an unhydrogenized styrene butadiene block copolymer (A2), and the hot-melt adhesive agent does not comprise an α-methyistyrene class resin (B) in an amount of not less than 5 parts by weight based on 100 parts by weight of the total weight of (A) and (B).

### CROSS-REFERENCE TO RELATED APPLICATIONS

This application is a continuation of International Patent Application No. PCT/JP2017/036274 filed Oct. 5, 2017, which claims the benefit of Japanese Patent Application No. 2016-209655 filed Oct. 26, 2016, the contents of both of which are incorporated herein by reference.

### FIELD OF THE INVENTION

The present invention relates to a hot-melt adhesive agent, and more particularly, to a hot-melt adhesive agent suitable for fixing an absorbent article such as a sanitary napkin to clothes such as a panty.

### BACKGROUND OF THE INVENTION

The absorbent article is an article used by being fixed to clothes such as underwear and a shirt, for example, to absorb bodily fluid coming out from a body, such as menstrual blood, vaginal discharge, urine and sweat, for example, and used for preventing a discomfort feeling and an unsanitary state caused by attachment of bodily fluid to clothes. As absorbent articles, known are sanitary napkins, urine absorbing liners, puerperant panties, breast milk pads, armpit sweat absorbing pads, paper diapers, pet sheets, hospital gowns and surgical white garments. Accordingly, an adhesive agent for positioning has also been developed for fixing such an absorbent article to clothes described above to prevent it from shifting from the position.

Many absorbent articles generally have an absorber between a moisture-permeable top sheet coming into contact with a skin and a liquid-impermeable back sheet coming into contact with underwear. An adhesive agent for positioning is generally applied to a release substrate material and then transferred to the back sheet. By bringing the transferred adhesive agent for positioning into contact with underwear, the absorbent article is retained to the underwear.

When the performance of an adhesive agent for positioning is insufficient, an absorbent article may not be retained to underwear causing bodily fluid to be attached to clothes. In recent years, therefore, there has been a demand for further improvement of the performance of adhesive agents for positioning.

For example, Patent Document 1 discloses a hot-melt adhesive agent using a hydrogenized block copolymer such as styrene-ethylene/butylene-styrene block copolymer (SEBS) as an adhesive agent for positioning (see claims). However, the hot-melt adhesive agent in Patent Document 1 has an aspect of being insufficient in retention strength, causing an absorbent article to shift from an adherend in some cases. In order to prevent shifting of the absorbent article completely, a comparatively large amount of the hot-melt adhesive agent is used.

A hot-melt adhesive agent in Patent Document 2 includes an unhydrogenized thermoplastic block copolymer and an α-methylstyrene class resin, and is suitable for a use of fixing an absorbent article such as a sanitary napkin to underwear ([Claim 1], [0001], [Table 1] to [Table 3]). The hot-melt adhesive agent in this document may prevent occurrence of residual adhesive but has an aspect of being insufficient in retention strength, causing the napkin to slightly shift from the adherend in some rare cases.

The hot-melt adhesive agents in Patent Documents 1 and 2 have room for improvement in retention strength, e.g., peel strength under an environment of low temperature (on the order of −10 to 10° C.).

In recent years, in order to reduce the odor of an absorbent particle, a fragrant material such as limonene is sometimes applied to the absorber. Since a fragrant material is volatile, it infiltrates inside a hot-melt adhesive agent applied to the back sheet, thereby reducing tack of the hot-melt adhesive agent. With reduction in the tack of the hot-melt adhesive agent, the hot-melt adhesive agent becomes so soft that the adhesive tends to be left behind on the adherend when the absorbent article is peeled off from the adherend.

In Patent Document 2, an α-methylstyrene class resin is blended in the hot-melt adhesive agent to improve the tack to the adherend. However, since the α-methylstyrene class resin includes a volatile component, the odor of this component may be felt unpleasant.

### CITATION LIST

[Patent Document 1] Japanese Patent Laid-open Publication No. 2008-297441

[Patent Document 2] Japanese Patent Laid-open Publication No. 2012-12437

### SUMMARY OF THE INVENTION

In order to solve the background art problems described above, an object of the present invention is providing a hot-melt adhesive agent that has excellent retention strength and anti-fragrance property and shows little residual adhesive and unpleasant odor.

The present invention and preferred embodiments of the present invention are as follows.

1. A hot-melt adhesive agent having a thermoplastic block copolymer (A), the (A) being a copolymer of vinyl class aromatic hydrocarbons and conjugated diene compounds, wherein

the thermoplastic block copolymer (A) comprises an unhydrogenized triblock type styrene isoprene block copolymer (A1) and an unhydrogenized styrene butadiene block copolymer (A2), and

the hot-melt adhesive agent does not comprise an α-methylstyrene class resin (B) in an amount of not less than 5 parts by weight based on 100 parts by weight of the total weight of (A) and (B).

2. The hot-melt adhesive agent according to 1, wherein the unhydrogenized triblock type styrene isoprene block copolymer (A1) has a styrene content of 10 to 60% by weight.

3. The hot-melt adhesive agent according to 1 or 2, comprising 5 to 50 parts by weight of the unhydrogenized triblock type styrene isoprene block copolymer (A1) based on 100 parts by weight of the total weight of the thermoplastic block copolymer (A).

4. The hot-melt adhesive agent according to any of 1 to 3, further comprising the α-methylstyrene class resin (B), the amount of (B) being not more than 4.5 parts by weight based on 100 parts by weight of the total weight of (A) and (B).

5. The hot-melt adhesive agent according to any of 1 to 4, further comprising a tackifying resin (C), the tackifying resin (C) including an unhydrogenized resin.

6. The hot-melt adhesive agent according to any of 1 to 5, wherein the hot-melt adhesive agent is used for positioning an absorbent article.

7. An absorbent article coated with the hot-melt adhesive agent according to any of 1 to 6.

The hot-melt adhesive agent of the present invention is capable of retaining an absorbent article such as a sanitary napkin to clothes such as underwear without shifting, does not leave residual adhesive behind on clothes when the absorbent article is peeled off from the clothes, has no odor and is free from reduction in performance due to a fragrant material such as limonene.

Accordingly, the hot-melt adhesive agent of the present invention is capable of improving the performance and usability of an absorbent article such as a sanitary napkin.

### BRIEF DESCRIPTION OF THE DRAWINGS

FIG. 1 is a plan view of a hot-melt adhesive agent molded body left at rest for 3 days under a 50° C. environment where limonene steam is present, showing that the anti-fragrance property is ⊚ with no distortion of the shape at all.

FIG. 2 is a plan view of a hot-melt adhesive agent molded body left at rest for 3 days under a 50° C. environment where limonene steam is present, showing that the anti-fragrance property is ∘ with little distortion of the shape.

FIG. 3 is a plan view of a hot-melt adhesive agent molded body left at rest for 3 days under a 50° C. environment where limonene steam is present, showing that the anti-fragrance property is X with distortion of the shape.

### DETAILED DESCRIPTION OF THE INVENTION

The hot-melt adhesive agent of the present invention has a thermoplastic block copolymer (A).

In the present invention, the “thermoplastic block copolymer (A)” refers to a copolymer where vinyl class aromatic hydrocarbons and conjugated diene compounds are block-copolymerized, and normally is a resin composition including one comprised of a vinyl class aromatic hydrocarbon block and a conjugated diene compound block.

The “vinyl class aromatic hydrocarbon” as used herein refers to an aromatic hydrocarbon compound having a vinyl group, and specific examples thereof include styrene, o-methylstyrene, p-methylstyrene, p-tert-butylstyrene, 1,3-dimethylstyrene, α-methylstyrene, vinylnaphthalene and vinylanthracene. Especially, styrene is preferable. These vinyl class aromatic hydrocarbons may be used alone or in combination.

The “conjugated diene compound” as used herein refers to a diolefin compound having at least a pair of conjugated double bonds. Specific examples of the “conjugated diene compound” include 1,3-butadiene, 2-methyl-1,3-butadiene (or isoprene), 2,3-dimethyl-1,3-butadiene, 1,3-pentadiene and 1,3-hexadiene. Especially preferred are 1,3-butadiene and 2-methyl-1,3-butadiene. These conjugated diene compounds may be used alone or in combination.

The thermoplastic block copolymer (A) of the present invention includes an unhydrogenized triblock type styrene isoprene block copolymer (A1) and an unhydrogenized styrene butadiene block copolymer (A2).

The unhydrogenized triblock type styrene isoprene block copolymer (A1) is a triblock type styrene isoprene block copolymer where a block based on a conjugated diene compound has not been hydrogenized, specifically a styrene-isoprene-styrene polymer (hereinafter referred to as “SIS”).

The unhydrogenized styrene butadiene block copolymer (A2) is a styrene butadiene block copolymer where a block based on a conjugated diene compound has not been hydrogenized, and may be a triblock type block copolymer or any of other type block copolymers (a radial type block copolymer or a linear type block copolymer).

In this specification, whether or not the thermoplastic block copolymer (A) has been hydrogenized is indicated by a “hydrogenation ratio.” When the hydrogenation ratio is 0%, the thermoplastic block copolymer (A) is an unhydrogenized copolymer.

The “hydrogenation ratio” of the thermoplastic block copolymer (A) refers to a proportion of double bonds hydrogenized and converted to saturated hydrocarbon bonds in all aliphatic double bonds included in a block based on a conjugated diene compound as the reference. This “hydrogenation ratio” may be measured with an infrared spectrophotometer and a nuclear magnetic resonance apparatus and the like.

In this specification, the triblock type styrene block copolymer refers to a styrene block copolymer that has a block represented by formula (1) below and does not include any other block. This is definitely distinguished from a linear type styrene block copolymer to be described later.

Chem. 1

S-E-S (1)

In formula (1), S is a styrene block and E is a conjugated diene block.

The linear type styrene block copolymer is a styrene block copolymer having both a block represented by formula (1) and a block represented by formula (2) below.

Chem. 2

S-E (2)

In formula (2), S and E have the same meanings as above.

The styrene conjugated diene block copolymer of formula (2) is called a “diblock” in some cases. The proportion of the styrene conjugated diene compound block copolymer of formula (2) included in the styrene block copolymer is referred to as a “diblock content”.

In other words, the triblock type styrene block copolymer has a diblock content of 0% by weight, and is definitely distinguished from the linear type styrene block copolymer by the diblock content.

In this specification, SIS (A1) has an isoprene block as E (conjugated diene block) in formula (1), and (A2) has both structures of formula (1) and formula (2) and has a butadiene block as E (conjugated diene block).

Since having SIS (A1), the hot-melt adhesive agent of the present invention improves in cohesion force thereby having excellent retention strength, has excellent anti-fragrance property, and when used for a sanitary napkin, is capable of reducing occurrence of residual adhesive.

SIS (A1) preferably has a styrene content of 10 to 60% by weight, more preferably 15 to 45% by weight, especially preferably 25 to 45% by weight. With a styrene content of (A1) within the above range, the anti-fragrance property of the hot-melt adhesive agent of the present invention further improves.

According to the present invention, SIS (A1) is preferably included by 15 to 50 parts by weight based on 100 parts by weight of the thermoplastic block copolymer (A).

With SIS (A1) included in the above proportion, the hot-melt adhesive agent of the present invention reduces occurrence of residual adhesive while maintaining excellent retention strength and also is excellent in anti-fragrance property and coating property, and therefore is especially suitable as a hot-melt adhesive agent for positioning a sanitary napkin.

Examples of commercial products of SIS (A1) include Vector 4411A (trade name), Vector 4211A (trade name), Vector 4211N (trade name) and Vector 4114N (trade name) manufactured by Dexco Polymers) and Kraton D1162PT manufactured by Kraton Performance Polymers, Inc.

Since having the unhydrogenized styrene butadiene block copolymer (A2), the hot-melt adhesive agent of the present invention improves in cohesion force thereby having excellent retention strength.

The unhydrogenized styrene butadiene block copolymer (A2) may be of the triblock type, the linear type or the radial type.

A specific structure of a radial type styrene block copolymer is represented by formula (3).

Chem. 3

(S-B)ₙY (3)

In formula (3), n is an integer equal to or more than 2, S is a styrene block, B is a butadiene block, and Y is a coupling agent. Preferably, n is 3 or 4, especially preferably 3. The above copolymer where n is 3 is referred to as a 3-branch type copolymer, and the above copolymer where n is 4 is referred to as a 4-branch type copolymer.

The coupling agent is a polyfunctional compound that couples linear type styrene block copolymers radially. The kind of the coupling agent is not particularly limited.

Examples of the coupling agent include: silane compounds such as halogenated silane and alkoxysilane; tin compounds such as halogenated tin; epoxy compounds such as polycarboxylates and epoxidized soybean oil; acryl esters such as pentaerythritol tetraacrylate; and divinyl compounds such as epoxysilane and divinylbenzene. As specific examples there are trichiorosilane, tribromosilane, tetrachlorosilane, tetrabromosilane, methyltrimethoxysilane, ethyltrimethoxysilane, vinyltrimethoxysilane, vinyltriethoxysilane, tetramethoxysilane, tetraethoxysilane, tetrachloro tin and diethyl adipate.

As the unhydrogenized styrene butadiene block copolymer (A2), the followings are commercially available: JSR TR2000 (trade name) and JSR TR2003 manufactured by JSR Corporation and Asaprene T420 (trade name), Asaprene T438 and Asaprene T439 (trade name) manufactured by Asahi Kasei Corporation.

According to the present invention, the thermoplastic block copolymer (A) may include other styrene block copolymers (A3) that correspond to neither (A1) nor (A2). Examples of the other styrene block copolymers (A3) include a “linear type styrene isoprene block copolymer,” a “radial type styrene isoprene block copolymer” and a “hydrogenized styrene block copolymer.”

Examples of commercial products of the linear type styrene isoprene block copolymer include Quintac 3270 (trade name), Quintac 3421 (trade name), Quintac 3433N (trade name) and Quintac 3520 (trade name) manufactured by Zeon Corporation.

Examples of the radial type styrene isoprene block copolymer include Quintac 3460 (trade name) and Quintac 3450 (trade name) manufactured by Zeon Corporation.

The hydrogenized styrene block copolymer is a block copolymer obtained by block-copolymerizing vinyl class aromatic hydrocarbons and conjugated diene compounds and hydrogenizing all or part of a block based on the conjugated diene compounds in the obtained block copolymer.

Examples of the hydrogenized styrene class triblock copolymer include a SEPS triblock copolymer, a SEBS triblock copolymer, a SEEPS triblock copolymer and a SEEBS triblock copolymer.

The SEPS triblock copolymer is a block copolymer comprised of an end block of a styrene polymer block and a middle block having an ethylene structure and a propylene structure in a mixed state, i.e., a styrene-ethylene/propylene-styrene copolymer.

The SEBS triblock copolymer is a block copolymer comprised of an end block of a styrene polymer block and a middle block having an ethylene structure and a butylene structure in a mixed state, i.e., a styrene-ethylene/butylene-styrene copolymer.

SEEPS is a block copolymer comprised of a styrene end block and a middle block of hydrogenated isoprene/butadiene.

Examples of commercial products of SEPS include SEPTON 2006, 2007, 2063, 2104, 4033 and 4044 (trade name) manufactured by Kuraray Co., Ltd.

Examples of commercial products of SEEPS include SEPTON 4099, 4077 and 4055 (trade name) manufactured by Kuraray Co., Ltd.

Examples of commercial products of SEBS include Kraton G1657, G1650, G1654 and G1651 (trade name) manufactured by Kraton Performance Polymers, Inc. and SEPTON 8007, 8076 and 8104 manufactured by Kuraray Co., Ltd.

The hot-melt adhesive agent of the present invention may include an α-methylstyrene class resin (B) blended into the thermoplastic block copolymer (A). However, in a preferred embodiment, the α-methylstyrene class resin (B) is not blended in an amount of not less than 5 parts by weight based on 100 parts by weight of the total weight of (A) and (B). The blending amount of the α-methylstyrene class resin (B) is preferably not more than 4.5 parts by weight, most desirably not more than 4 parts by weight.

With the α-methylstyrene class resin (B) blended in an amount within the above range, the hot-melt adhesive agent of the present invention has excellent anti-fragrance property and suppresses generation of odor of a volatile material.

As the α-methylstyrene class resin (B), an α-methylstyrene copolymer or a styrene-α-methylstyrene copolymer is used. As an embodiment of the present invention, the α-methylstyrene class resin (B) is more preferably a styrene-α-methylstyrene copolymer. Especially, that having a softening point of 85 to 120° C. (measured by the ring and ball method stipulated in JIS K2207) is more preferable. Specific examples of commercial products of (B) include Crystalex 3085 (trade name), Crystalex 3100 (trade name), Crystalex 1120 (trade name) and Crystalex 5140 (trade name) manufactured by Eastman Chemical Company and FTR-2120 (trade name) manufactured by Mitsui Chemicals, Inc.

It should be noted that in this specification, the α-methylstyrene class resin is not included in a tackifying resin (C) to be described later.

The hot-melt adhesive agent of the present invention preferably has a tackifying resin (C) and a plasticizer (D). The tackifying resin is not particularly limited as long as it is normally used for hot-melt adhesive agents and the hot-melt adhesive agent as the object of the present invention is obtainable.

According to the present invention, the addition amount of the tackifying resin (C) is preferably 10 to 250 parts by weight based on 100 parts by weight of the total weight of (A) and (B).

When the addition amount of the tackifying resin (C) is within the above range, it is possible to reduce the addition amount of the α-methylstyrene class resin (B) in the hot-melt adhesive agent of the present invention. Therefore, the odor become low, and moreover the anti-fragrance property improves.

Examples of the tackifying resin (C) include natural rosin, modified rosin, hydrogenized rosin, glycerol ester of natural rosin, glycerol ester of modified rosin, pentaerythritol ester of natural rosin, pentaerythritol ester of modified rosin, pentaerythritol ester of hydrogenized rosin, a copolymer of natural terpene, a three-dimensional polymer of natural terpene, a hydrogenated derivative of a copolymer of hydrogenized terpene, a polyterpene resin, a hydrogenated derivative of a phenol class modified terpene resin, an aliphatic petroleum hydrocarbon resin, a hydrogenated derivative of an aliphatic petroleum hydrocarbon resin, an aromatic petroleum hydrocarbon resin, a hydrogenated derivative of an aromatic petroleum hydrocarbon resin, a cycloaliphatic petroleum hydrocarbon resin and a hydrogenated derivative of a cycloaliphatic petroleum hydrocarbon resin. These tackifying resins may be used alone or in combination. A liquid type tackifying resin may also be used as long as it has a color tone of colorless to pale yellow, has substantially no odor and is good in thermal stability. In consideration of these properties comprehensively, an unhydrogenized tackifying resin is preferable as the tackifying resin.

As the tackifying resin (C), a commercial product can be used. Examples of such commercial products include ECR 179EX (trade name) manufactured by Tonex Co., Ltd., Marca Clear H (trade name) manufactured by Maruzen Petrochemical Co., Ltd., Arcon M100 (trade name) manufactured by Arakawa Chemical Industries, Ltd., I-MARV S100 (trade name) manufactured by Idemitsu Kosan Co., Ltd., Clearon K100 (trade name), Clearon K4090 (trade name) and Clearon K4100 manufactured by Yasuhara Chemical Co., Ltd., ECR 179EX (trade name) and ECR 231C (trade name) manufactured by Tonex Co., Ltd., Regalite C6100L (trade name) and Regalite C8010 (trade name) manufactured by Eastman Chemical Company, FTR 2140 (trade name) manufactured by Mitsui Chemicals, Inc. and Quintone DX390N (trade name) and Quintone DX395 (trade name) manufactured by Zeon Corporation. These commercial tackifying resins may be used alone or in combination.

According to the present invention, by use of an unhydrogenized tackifying resin, it is possible to obtain a hot-melt adhesive agent having little residual adhesive and excellent retention strength. As an unhydrogenized tackifying resin, Quintone DX390N (trade name) or Quintone DX395 (trade name) may be used to obtain a hot-melt adhesive agent excellent in the balance between residual adhesive and retention strength.

According to the present invention, the addition amount of the tackifying resin (C) is preferably 10 to 200 parts by weight, especially desirably 130 to 200 parts by weight, further desirably 150 to 190 parts by weight, most desirably 170 to 190 parts by weight, based on 100 parts by weight of the total weight of (A) and (B). With the blending amount of the tackifying resin (C) within the above range, the hot-melt adhesive agent of the present invention is provided with tack, permitting prevention of occurrence of residual adhesive.

The plasticizer (D) is blended for the purpose of reducing melt viscosity of the hot-melt adhesive agent, imparting flexibility to the hot-melt adhesive agent and improving wetting to the adherend, and is not particularly limited as long as it is compatible with a block copolymer and the hot-melt adhesive agent as the object of the present invention is obtainable. Examples of the tackifying resin (D) include paraffin class oil, naphthene class oil and aromatic oil. Paraffin class oil that is colorless and odorless is especially preferable.

As the plasticizer (D), a commercial product may be used. Examples of such commercial products include White Oil Broom 350 (trade name) manufactured by Kukdong Oil & Chem, Diana Fresia S32 (trade name), Diana Process Oil PW-90 (trade name) and DN Oil KP-68 (trade name) manufactured by Idemitsu Kosan Co., Ltd., Enerper M1930 (trade name) manufactured by BP Chemicals, Ltd., Kaydol (trade name) manufactured by Crompton Corp., Primo 1352 (trade name) manufactured by Exxon Mobile Corp., Process Oil NS100 manufactured by Idemitsu Kosan Co., Ltd., KN4010 (trade name) manufactured by PetroChina Co., Ltd. and Sunpure N90 manufactured by Sun Oil Co., Ltd. These plasticizers (D) may be used alone or in combination.

The hot-melt adhesive agent of the present invention may further include a stabilizer (E). The “stabilizer” is blended for the purpose of improving the stability of the hot-melt adhesive agent by preventing heat-causing reduction in molecular weight, gelation, coloring and generation of odor, and is not particularly limited as long as the hot-melt adhesive agent as the object of the present invention is obtainable. Examples of the “stabilizer” include an antioxidant and an ultraviolet absorber.

The “ultraviolet absorber” is used to improve the light resistance of the hot-melt adhesive agent. The “antioxidant” is used to prevent oxidation degradation of the hot-melt adhesive agent. The antioxidant and the ultraviolet absorber are generally used for disposable products, and are not particularly limited as long as target disposable products to be described later are obtainable.

Examples of the antioxidant include phenol class antioxidants, sulfur class antioxidants and phosphorous class antioxidants. Examples of the ultraviolet absorber include benzotriazole class ultraviolet absorbers and benzophenone class ultraviolet absorbers. It is also possible to add lactone class stabilizers. These can be used alone or in combination.

As the stabilizer (E), commercial products can be used. Examples thereof include SUMILIZER GM (trade name), SUMILIZER TPD (trade name) and SUMILIZER TPS (trade name) manufactured by Sumitomo Chemical Co., Ltd., IRGANOX 1010 (trade name) and IRGANOX HP2225FF (trade name) and IRGAFOS 168 (trade name) manufactured by Ciba Specialty Chemicals, Inc. and JF77 (trade name) manufactured by Johoku Chemical Co., Ltd. These can be used alone or in combination. The stabilizer is generally used for disposable products, and is not particularly limited as long as target disposable products to be described later are obtainable.

The hot-melt adhesive agent of the present invention may include an additive other than those described above, such as a fine particle filler, as required.

The hot-melt adhesive agent of the present invention is manufactured by blending the above components in a predetermined proportion, further blending various additives as required, and melt-mixing the blend by heating. Specifically, the hot-melt adhesive agent is manufactured by putting the above components in a melt-mixing vessel equipped with a stirrer and heat-mixing them.

The obtained hot-melt adhesive agent preferably has a melt viscosity at 160° C. of not more than 5,000 mPas, especially preferably 2,000 to 5,000 mPas, most preferably 3,000 to 4,500 mPas

In the present description, the “melt viscosity” refers to the viscosity of the hot-melt adhesive agent in a molten state, which is measured with a Brookfield RVT type viscometer (spindle No. 27).

The hot-melt adhesive agent of the present invention, having a melt viscosity at 160° C. as low as not more than 5,000 mPas, is excellent in coating performance.

The hot-melt adhesive agent of the present invention may be fed in a liquid or semi-liquid form. Since the hot-melt adhesive agent may be stored in an insulating container in a liquid or semi-liquid form and fed as it is, it is also very preferable from an environmental standpoint. That is, it is possible to reduce generation of waste materials and save power consumption in manufacture of the hot-melt adhesive agent.

Moreover, the hot-melt adhesive agent of the present invention preferably has a retention strength at 40° C. of not less than 10 minutes, more preferably not less than 30 minutes, especially preferably not less than 50 minutes, in an evaluation method for retention strength to be described in Examples.

The hot-melt adhesive agent of the present invention preferably has a peel strength (10° C., 20° C., 40° C.) of not less than 1,200 gf/inch (11.8 N/3.05 cm), more preferably 1,400 gf/inch (13.7 N/3.56 cm), in an evaluation method for peel strength to be described in Examples.

The hot-melt adhesive agent of the present invention may be manufactured by blending (A) and (B), and further blending various additives as required, by a generally known manufacturing method for hot-melt adhesive agents. For example, it may be manufactured by blending the above components by predetermined amounts and melting them by heating. The order of addition of the components and the heating method are not particularly limited as long as the target hot-melt adhesive agent is obtainable.

According to the present invention, using a dynamic viscoelasticity measuring instrument, a storage elastic modulus G′ is measured with each velocity fixed to 10 rad/s, scanning the temperature in a range of −25° C. to 150° C. at a temperature rise rate of 5° C./minute in a temperature sweep mode, to obtain a scanned value at 50° C. While the storage elastic modulus G′ is a property value corresponding to elasticity, a loss elastic modulus G″ is known as a property value corresponding to viscosity. Since G″ is measured simultaneously with measurement of G′ with the dynamic viscoelasticity measuring instrument, a loss tangent (tan delta) indicated by the ratio of loss elastic modulus G″ to storage elastic modulus G′ (G″/G′) may be obtained simultaneously.

The hot-melt adhesive agent of the present invention preferably has a glass transition temperature (Tg) of not more than 15° C., more preferably −10° C. to 13° C., most desirably 0° C. to 10° C. The hot-melt adhesive agent, having a glass transition temperature (Tg) falling within the above range, is kept from becoming too hard, and capable of firmly retaining an absorbent article to underwear even in a cool region. If Tg of the hot-melt adhesive agent is high compared with the use environment temperature of the absorbent article, the hot-melt adhesive agent changes its state to a glass state, causing difficulty in retaining the absorbent article to underwear with the hot-melt adhesive agent. Considering use of the absorbent article in a cool region, Tg of the hot-melt adhesive agent is preferably within the above range.

As used herein, the glass transition temperature (Tg) refers to a temperature indicating the peak top of a peak obtained by blotting, with respect to the temperature, the loss tangent (tan delta) measured simultaneously with the measurement of the storage elastic modulus G′ performed with the frequency fixed to 10 Rad/s using the above dynamic viscoelasticity measuring instrument.

In the case of applying the hot-melt adhesive agent to a sanitary napkin having a polyolefin film as the back sheet, it is possible to directly apply the hot-melt adhesive agent to the polyolefin film easily because the viscosity at 160° C. is not more than 5,000 mPas. With the ability of being applied uniformly to the film easily at 160° C., the hot-melt adhesive agent is free from degradation, and the sanitary napkin is firmly retained to underwear and, when being peeled off from the underwear, prevented from occurrence of residual adhesive on the underwear.

While the hot-melt adhesive agent of the present invention is widely used for paper processing, bookbinding and disposable products, it is especially effectively used for absorbent articles. The “absorbent articles” are not particularly limited as long as they are so-called sanitary materials. Specific examples thereof include sanitary napkins, urine absorbing liners, puerperant panties, breast milk pads, armpit sweat absorbing pads, paper diapers, pet sheets, hospital gowns and surgical white garments.

The absorbent article is constituted by applying the hot-melt adhesive agent of the present invention to at least one kind of members selected from the group consisting of woven fabric, nonwoven fabric, rubber, resin, paper and a polyolefin film. As the polyolefin film, a polyethylene film is preferable for the reasons of durability and cost.

The hot-melt adhesive agent of the present invention is suitable for a sanitary napkin. In general, a sanitary napkin has an absorber between a top sheet and a back sheet, for example, with the top sheet coming into contact with a skin and the back sheet coming into contact with underwear. The hot-melt adhesive agent of the present invention exerts its effect especially when the back sheet of the sanitary napkin not in contact with a skin is a polyolefin film.

By increasing the peel strength between the polyolefin film coated with the hot-melt adhesive agent and the underwear, the sanitary napkin is prevented from shifting from the underwear. According to the present invention, the hot-melt adhesive agent may be directly applied to the polyolefin film, or may be first applied to a release film, which may then be transferred to the polyolefin film, whereby it is possible to prevent not only shifting from the underwear but also occurrence of residual adhesive on the underwear.

In a manufacturing line for absorbent articles, the hot-melt adhesive agent is generally applied to any of various members (e.g., release paper, a tissue, cotton, nonwoven fabric and a polyolefin film and the like) of disposable products. In application, the hot-melt adhesive agent may be ejected from various ejectors.

The method for coating with the hot-melt adhesive agent is not particularly limited as long as target absorbent articles are obtainable. Such coating methods are largely classified into contact coating and non-contact coating. The “contact coating” refers to a coating method where an ejector is brought into contact with a member or a film at coating with the holt-melt adhesive agent, and the “non-contact coating” refers to a coating method where an ejector is not brought into contact with a member or a film at coating with the holt-melt adhesive agent. Examples of the contact coating method include slot coating and roll coating. Examples of the non-contact coating include spiral coating capable of coating in a spiral form, omega coating and control seam coating capable of coating in a wavy form, slot spray coating and curtain spray coating capable of coating in a plane form and dot coating capable of coating in a dot form.

The hot-melt adhesive agent is applied by any of the above coating methods, to manufacture an absorbent article. Examples of the absorbent article include sanitary napkins, urine absorbing liners, puerperant panties, breast milk pads, armpit sweat absorbing pads, paper diapers, pet sheets, hospital gowns and surgical white garments. According to the present invention, the effect can be most exerted on a sanitary napkin with a polyolefin film as the back sheet.

### Examples

The present invention will be described by way of example for the purpose of describing the present invention in more detail and in more specific manner. These examples are only for illustration and by no means intended to restrict the present invention.

The components for preparation of the hot-melt adhesive agent are shown below.

(A) Thermoplastic block copolymer that is a copolymer of vinyl class aromatic hydrocarbons and conjugated diene compounds

(A1-1) Unhydrogenized triblock type styrene isoprene block copolymer (Kraton D1162PT (trade name) manufactured by Kraton Performance Polymers, Inc.)

(A1-2) Unhydrogenized triblock type styrene isoprene block copolymer (Vector 4211N (trade name) manufactured by Dexco Polymers)

(A1-3) Unhydrogenized triblock type styrene isoprene block copolymer (Vector 4114N (trade name) manufactured by Dexco Polymers)

(A2) Unhydrogenized styrene butadiene diblock copolymer (Asaprene T438 (trade name) manufactured by Asahi Kasei Corporation)

(A3-1) Unhydrogenized linear type styrene isoprene block copolymer (Quintac 3520N (trade name) manufactured by Zeon Corporation)

(A3-2) Unhydrogenized linear type styrene isoprene block copolymer (Quintac 3433N (trade name) manufactured by Zeon Corporation)

(B) α-methylstyrene class resin

(B1) Styrene-α-methylstyrene copolymer (Crystalex 3100 (trade name) manufactured by Eastman Chemical Company)

(C) Tackifying resin

(C1) Hydrogenated derivative of aromatic petroleum hydrocarbon resin (Quintone DX395 (trade name) manufactured by Zeon Corporation)

(C2) Hydrogenated derivative of cycloaliphatic petroleum hydrocarbon resin (Escorets HA103 (trade name) manufactured by Exxon Mobile Corp.)

(D) Plasticizer

(D1) Paraffin class oil (Diana Fresia S-32 (trade name) manufactured by Idemitsu Kosan Co., Ltd.)

(E) Stabilizer

(E1) Hindered phenol class antioxidant (IRGANOX 1010 (trade name) manufactured by Ciba Specialty Chemicals, Inc.)

(E2) Benzotriazole class ultraviolet absorber (Chisorb P (trade name) manufactured by Double Bond Chemical Ind., Co., Ltd.)

The blending ratio of the components (A) to (E) is shown in Tables 1 to 3. The components were melt-mixed at approximately 140° C. over 2 hours using a universal stirrer, to manufacture hot-melt adhesive agents of Examples 1 to 13 and Comparative Examples 1 to 5. All the figures related to the composition (blending) of the hot-melt adhesive agents shown in Tables 1 to 3 represent parts by weight.

The glass transition temperature was measured for the hot-melt adhesive agents of the examples and the comparative examples, to evaluate the anti-fragrance property, the retention strength, the residual adhesive and the odor. The summary of the evaluations will be described hereinafter.

<Retention Strength (Peel Strength)>

Each of the hot-melt adhesive agents was applied to a PET film having a thickness of 50 μm, to form an adhesive layer having a thickness of 50 μm on the PET film. The coated PET film was formed into a 25 mm wide shape to obtain a specimen.

Silk for JIS dye fastness test (in conformity with JIS L0803) was cut into a size of 30×70 mm in the folding line direction, to be used as a substrate material for bonding.

The specimen and the substrate material for bonding were cured under a 23° C. environment for not less than 30 minutes, and then bonded together with a 2 kg roller. Immediately after the bonding, a 180° peel test was performed using a universal tensile testing machine at a speed of 300 mm/min. At least 3 specimens were measured for each hot-melt adhesive agent, and the average thereof was determined as the value of the peel strength.

The peel strength was evaluated based on the following criteria.
⊚ . . . more than 660/26 mm
∘ . . . 400 to 660/25 mm
Δ . . . 200 to 400/25 mm
X . . . less than 200/25 mm

<Residual Adhesive>

Immediately after the measurement of the peel strength, the peeled surface of the substrate material for bonding was checked by finger tough, to evaluate presence/absence of residual adhesive. Since silk was found to be a substrate material on which residual adhesive was remarkably detectable, silk was used as the evaluation substrate material. The evaluation criteria for residual adhesive are as follows.
⊚ . . . There is no residual adhesive
∘ . . . There is slightly residual adhesive
Δ . . . There is some residual adhesive
X . . . There is clearly residual adhesive

<Anti-Fragrance Property>

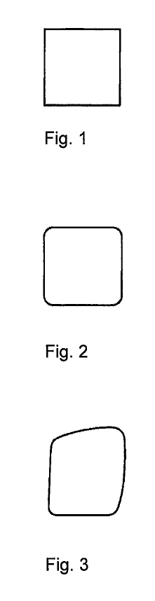

Each hot-melt adhesive agent is formed into a shape of a cube (5 mm×5 mm×5 mm) to obtain a specimen. One face of the specimen is bonded to a PET film, and such specimens were placed on the bottom of a sealing container (volume: 710 ml, metal tube). Immediately after 0.4 ml of D-limonene (Yasuhara Chemical Co., Ltd.) was dropped into a glass bottle (15 ml) and the specimens were fixed to the bottom of the sealing container, the lid of the container was closed, and the container was left at rest under a 50° C. environment for 3 days. As a blank (control sample), the test was also performed without dropping of D-limonene, and the container was also left at rest under a 50° C. environment for 3 days. Thereafter, how the shapes of the specimens were changed was observed.
⊚ . . . The shape is maintained with no difference from control sample (see FIG. 1).
∘ . . . Although there is some difference from control sample, the shape is maintained (see FIG. 2; the corners of the cube are somewhat round).
X . . . There is difference from control sample, and the shape is distorted (see FIG. 3 the shape of the cube is distorted into a round).

<Odor>

Each hot-melt adhesive agent, 30 g, was weighed in a glass bottle (70 ml), melted at 180° C. in a dryer and cooled. After the cooling, the sample was cured at 40° C. for 30 minutes in the dryer, to prepare a specimen. The odor of the specimen was evaluated by 10 subjects, and the odor level was artificially converted to a numerical form.

The following evaluation criteria were set, determining the numbers of the odor level based on the subjects' senses.

Odor level (sensory evaluation)
1 . . . No odor is sensed.
2 . . . Odor is slightly sensed.
3 . . . Odor is discernible.
4 . . . Odor is sensed.
5 . . . Strong odor is sensed.

For each hot-melt adhesive agent, the average of the odor levels of 10 subjects was calculated, and the odor of the hot-melt adhesive agent was evaluated by this average. The evaluation criteria are as follows.
⊚ . . . The average is less than 2.0.
∘ . . . The average is 2.0 to 3.0.
Δ . . . The average is 3.0 to 3.5.
X . . . The average exceeds 3.5.

<Glass Transition Temperature>

The glass transition temperature was measured using a dynamic viscoelasticity measuring instrument (RheometerAR-G2 (trade name) manufactured by TA Instruments). The hot-melt adhesive agent was heated with a jig of a device, to be formed into a disk having a diameter of 25 mm and a thickness of 15,000 μm.

The measurement of the glass transition temperature was performed using a parallel plate made of stainless steel with an angular velocity fixed to 10 rad/s, increasing the temperature at a rate of 5° C./minute in a range of −25° C. to 150° C. in a temperature sweep mode.

The loss tangent (tan delta) indicated by the ratio of the loss elastic modulus (G″) to the storage elastic modulus (G′) (G″/G′) is measured within a temperature range not more than the softening point. The temperature indicating the peak top of a peak obtained by blotting, with respect to the temperature, the loss tangent (tan delta), to determine the glass transition temperature of the hot-melt adhesive agent

TABLE 1

| | | Exam- | Exam- | Exam- | Exam- | Exam- | Exam- | Exam- |
|---|---|---|---|---|---|---|---|---|
| | | ple 1 | ple 2 | ple 3 | ple 4 | ple 5 | ple 6 | ple 7 |
| (A) Thermoplastic | (A1-1) Triblock type SIS (Kraton D1162PT, St40%) | 15 | 40 | 60 | 70 | 37 | 37 | 36 |
| block copolymer | (A1-2) Triblock type SIS (Vector 4211N, St30%) | | | | | | | |
| | (A1-3) Triblock type SIS (Vector 4114N, St15%) | | | | | | | |
| | (A2) Diblock type SB (Asaprene T438) | 85 | 60 | 40 | 30 | 60 | 60 | 60 |
| | (A3-1) Linear type SIS (Quintac 3520N) | | | | | | | |
| | (A3-2) Linear type SIS (Quintac 3433N) | | | | | | | |
| (B) α-methylstyrene | St-α-methyl St copolymer (Kristalex 3100) | 0 | 0 | 0 | 0 | 3.0 | 3.5 | 4 |
| class resin | | | | | | | | |
| (C) Tackifying resin | Unhydrogenized tackifying resin (Quintone DX395) | 180 | 180 | 180 | 180 | 180 | 180 | 180 |
| | Unhydrogenized tackifying resin (Escorets HA103) | | | | | | | |
| (D) Plasticizer | Paraffin class oil (Diana Fresia S-32) | 70 | 70 | 70 | 70 | 70 | 70 | 70 |
| (E) Stabilizer | Antioxidant (IRGANOX 1010) | 2 | 2 | 2 | 2 | 2 | 2 | 2 |
| | UV absorber (Chisorb P) | 1 | 1 | 1 | 1 | 1 | 1 | 1 |
| Performance | Anti-fragrance property | ◯ | ⊚ | ⊚ | ⊚ | ⊚ | ⊚ | ◯ |
| evaluation | Viscosity (160° C.) | 2500 | 3600 | 3680 | 4510 | 3180 | 3150 | 3200 |
| | Residual adhesive | ◯ | ◯ | ⊚ | ⊚ | ◯ | ◯ | ◯ |
| | Low-temperature peel strength | ◯ | ⊚ | ◯ | ◯ | ◯ | ◯ | ◯ |
| | Peel strength | ◯ | ⊚ | ⊚ | ◯ | ◯ | ⊚ | ⊚ |
| | Odor | ◯ | ◯ | ◯ | ◯ | ◯ | ◯ | ◯ |
| | Tg (° C.) | 9 | 8 | 9 | 9 | 7 | 7 | 8 |

TABLE 2

| | | Exam- | Exam- | Exam- | Exam- | Exam- | Exam- |
|---|---|---|---|---|---|---|---|
| | | ple 8 | ple 9 | ple 10 | ple 11 | ple 12 | ple 13 |
| (A) Thermoplastic | (A1-1) Triblock type SIS (Kraton D1162PT, St40%) | 40 | 40 | 40 | | | 40 |
| block copolymer | (A1-2) Triblock type SIS (Vector 4211N, St30%) | | | | 40 | | |
| | (A1-3) Triblock type SIS (Vector 4114N, St15%) | | | | | 40 | |
| | (A2) Diblock type SB (Asaprene T438) | 60 | 60 | 60 | 60 | 60 | 60 |
| | (A3-1) Linear type SIS (Quintac 3520N) | | | | | | |
| | (A3-2) Linear type SIS (Quintac 3433N) | | | | | | |
| (B) α-methylstyrene | St-α-methyl St copolymer (Kristalex 3100) | 0 | 0 | 0 | 0 | 0 | 0 |
| class resin | | | | | | | |
| (C) Tackifying resin | Unhydrogenized tackifying resin (Quintone DX395) | 140 | 160 | 185 | 180 | 180 | |
| | Unhydrogenized tackifying resin (Escorets HA103) | | | | | | 180 |
| (D) Plasticizer | Paraffin class oil (Diana Fresia S-32) | 110 | 90 | 65 | 70 | 70 | 70 |
| (E) Stabilizer | Antioxidant (IRGANOX 1010) | 3 | 3 | 2 | 2 | 2 | 2 |
| | UV absorber (Chisorb P) | 1 | 1 | 1 | 1 | 1 | 1 |
| Performance | Anti-fragrance property | ◯ | ⊚ | ◯ | ⊚ | ◯ | ⊚ |
| evaluation | Viscosity (160° C.) | 1800 | 2200 | 3900 | 3200 | 3030 | 3350 |
| | Residual adhesive | Δ | ◯ | ◯ | ◯ | ◯ | ◯ |
| | Low-temperature peel strength | Δ | ⊚ | ◯ | ⊚ | ⊚ | ◯ |
| | Peel strength | Δ | ◯ | ⊚ | ◯ | ◯ | ◯ |
| | Odor | ◯ | ◯ | ◯ | ◯ | ◯ | ⊚ |
| | Tg (° C.) | 5 | 7 | 13 | 11 | 9 | 11 |

TABLE 3

| | | Comparative | Comparative | Comparative | Comparative | Comparative |
|---|---|---|---|---|---|---|
| | | Example 1 | Example 2 | Example 3 | Example 4 | Example 5 |
| (A) Thermoplastic | (A1-1) Triblock type SIS (Kraton D1162PT, St40%) | | 100 | 35 | | |
| block copolymer | (A1-2) Triblock type SIS (Vector 4211N, St30%) | | | | | |
| | (A1-3) Triblock type SIS (Vector 4114N, St15%) | | | | | |
| | (A2) Diblock type SB (Asaprene T438) | 100 | | 60 | 60 | 60 |
| | (A3-1) Linear type SIS (Quintac 3520N) | | | | 40 | |
| | (A3-2) Linear type SIS (Quintac 3433N) | | | | | 40 |
| (B) α-methylstyrene | St-α-methyl St copolymer (Kristalex 3100) | 0 | 0 | 5 | 0 | 0 |
| class resin | | | | | | |
| (C) Tackifying resin | Unhydrogenized tackifying resin (Quintone DX395) | 180 | 180 | 180 | 180 | 180 |
| | Unhydrogenized tackifying resin (Escorets HA103) | | | | | |
| (D) Plasticizer | Paraffin class oil (Diana Fresia S-32) | 70 | 70 | 70 | 70 | 70 |
| (E) Stabilizer | Antioxidant (IRGANOX 1010) | 2 | 2 | 2 | 2 | 2 |
| | UV absorber (Chisorb P) | 1 | 1 | 1 | 1 | 1 |
| Performance | Anti-fragrance property | X | ⊚ | X | X | X |
| evaluation | Viscosity (160° C.) | 2010 | 4100 | 3200 | 3330 | 3820 |
| | Residual adhesive | X | ◯ | ◯ | Δ | Δ |
| | Low-temperature peel strength | ◯ | X | ◯ | Δ | ◯ |
| | Peel strength | ◯ | X | ⊚ | Δ | ◯ |
| | Odor | ◯ | ◯ | X | ◯ | ◯ |
| | Tg (° C.) | 8 | 10 | 8 | 11 | 12 |

As shown in Tables 1 and 2, each of the hot-melt adhesive agents of the examples include both the unhydrogenized triblock type styrene isoprene block copolymer (A1) and the unhydrogenized styrene butadiene block copolymer (A2), and also includes the α-methylstyrene class resin (B) in an amount of less than 5 parts by weight. Therefore, the hot-melt adhesive agent has no odor, is free from reduction in performance due to fragrance such as limonene, is excellent in peel strength and has little residual adhesive.

On the contrary, as shown in Table 3, each of the hot-melt adhesive agents of the comparative examples does not include the component (A1) or (A2), or includes the component (B) in an amount of not less than 5 parts by weight. Therefore, such a hot-melt adhesive agent is significantly inferior in any of the above properties to the hot-melt adhesive agents of the examples.

From the above results, it has been proved that the hot-melt adhesive agent including the components (A1) and (A2) and also including the component (B) in an amount of less than 5 parts by weight as required has no odor, is excellent in anti-fragrance property and retention strength and has little residual adhesive, and thus is especially effective for positioning of an absorbent article.

### INDUSTRIAL APPLICABILITY

The present invention is capable of providing a hot-melt adhesive agent and an absorbent article coated with the hot-melt adhesive agent. The absorbent article according to the present invention is especially effective as a sanitary napkin having a polyolefin film as the back sheet not in contact with a skin.

1: A hot-melt adhesive agent having a thermoplastic block copolymer (A), which is a copolymer of vinyl class aromatic hydrocarbons and conjugated diene compounds, and an α-methylstyrene class resin (B);
wherein the thermoplastic block copolymer (A) comprises an unhydrogenized triblock type styrene isoprene block copolymer (A1) and an unhydrogenized styrene butadiene block copolymer (A2), and
wherein the α-methylstyrene class resin (B) is present in an amount of not less than 5 parts by weight based on 100 parts by weight of the total weight of (A) and (B).

2: The hot-melt adhesive agent according to claim 1, wherein the unhydrogenized triblock type styrene isoprene block copolymer (A1) has a styrene content of 10 to 60% by weight.

3: The hot-melt adhesive agent according to claim 1, comprising 15 to 50 parts by weight of the unhydrogenized triblock type styrene isoprene block copolymer (A1) based on 100 parts by weight of the total weight of hot-melt adhesive agent (A).

4: The hot-melt adhesive agent according to claim 1, wherein the α-methylstyrene class resin (B) is present in an amount of less than 4.5 parts by weight.

5: The hot-melt adhesive agent according to claim 1, wherein the hot-melt adhesive agent is a positioning adhesive for an absorbent article.

6: An absorbent article coated with the hot-melt adhesive agent according to claim 1.
